(19) United States
(12) Patent Application Publication
(10) Pub. No.: US 2020/0309760 A1
(43) Pub. Date: Oct. 1, 2020

(54) SYSTEMS AND METHODS FOR IDENTIFYING MATERIAL DURING AN OPHTHALMIC PROCEDURE USING AC IMPEDANCE MEASUREMENT

(71) Applicant: Alcon Inc., Fribourg (CH)

(72) Inventor: Dan Durant, Rancho Santa Margarita, CA (US)

(21) Appl. No.: 16/826,828

(22) Filed: Mar. 23, 2020

Related U.S. Application Data
(60) Provisional application No. 62/824,347, filed on Mar. 27, 2019.

(51) Int. Cl. G01N 33/487; A61M 1/00; A61F 9/007; G01N 27/02; G01N 27/327

(52) U.S. Cl. CPC G01N 33/48707; A61M 1/0084; A61F 9/00745; A61M 2210/0612; G01N 27/028; G01N 27/3275; A61M 2230/65; A61M 1/0064

(57) ABSTRACT

Systems and methods are disclosed for identifying material aspirated during an ophthalmic procedure. Example systems comprise a first electrode and a second electrode positioned such that aspirated material flows between the first electrode and the second electrode and an alternating current impedance measuring component connected to the first electrode and the second electrode. The alternating current impedance measuring component measures the impedance of the aspirated material between the first electrode and the second electrode, and, based upon the measured impedance, the aspirated material can be identified.

### TECHNICAL FIELD

The present disclosure is directed to systems and methods for the identification of material aspirated during ophthalmic procedures.

### BACKGROUND

A number of different ophthalmic procedures are performed in which different materials are aspirated from the eye. For example, in vitreoretinal surgery, a device may be used to remove vitreous material from the eye. As another example, in cataract surgery, a device may be used to fragment or emulsify a lens and to remove the broken or emulsified lens from the eye. In these or other procedures, a balanced salt solution (BSS) may be introduced into the eye and removed during the procedure.

Currently, it can be difficult for a person performing an ophthalmic procedure to know what type of material is being aspirated from the eye at any given time during the procedure. Accordingly, a need exists for systems and methods for identifying materials aspirated during ophthalmic procedures.

### SUMMARY

The present disclosure is directed to systems and methods for identifying materials aspirated during ophthalmic procedures.

In some embodiments, a system for identifying material aspirated during an ophthalmic procedure comprises: (a) an aspiration path through which aspirated material flows away from the eye during the ophthalmic procedure, and (b) a circuit, wherein the circuit comprises: (i) a first electrode and a second electrode positioned such that aspirated material flows between the first electrode and the second electrode, and (ii) an alternating current (AC) impedance measuring component connected to the first electrode and the second electrode. The AC impedance measuring component measures the impedance of the aspirated material between the first electrode and the second electrode, and, based upon the measured impedance, the aspirated material type can be identified.

The first electrode and the second electrode together may take any suitable form allowing impedance measurement. In some embodiments, the first electrode and second electrode together with the aspirated material flowing between them may be analogous to a capacitor. In some embodiments, the first electrode may comprise a first flat plate and the second electrode may comprise a second flat plate parallel to the first flat plate. In some embodiments, the first electrode may comprise a tube and the second electrode may comprise a probe inside of the tube.

The AC impedance measuring component may be configured to supply alternating current through the electrodes. The AC impedance measuring component may be configured to supply alternating current through the electrodes at a frequency from approximately 200 kHz to approximately 5 MHz.

In some embodiments, the system comprises an ophthalmic instrument for performing an ophthalmic procedure, the ophthalmic instrument comprising an aspiration path through which aspirated material flows away from the eye during the ophthalmic procedure. The first electrode and the second electrode are positioned such that aspirated material flows between the first electrode and the second electrode when the aspirated material flows away from the eye, and the AC impedance measuring component measures the impedance of the aspirated material between the first electrode and the second electrode in order to identify the aspirated material. The ophthalmic instrument may comprise a vitrectomy instrument, a phacofragmentation handpiece, a phacoemulsification handpiece, an aspirating handpiece, and/or an extrusion handpiece.

In some embodiments, the first electrode and the second electrode are part of an ophthalmic instrument. In some embodiments, the first electrode and the second electrode are positioned outside of an ophthalmic instrument. In some embodiments, the first electrode and the second electrode are positioned inside of an aspiration tube.

In some embodiments, a method of identifying material aspirated during an ophthalmic procedure comprises: (a) aspirating material from an eye through an aspiration path, wherein aspirated material flows between a first electrode and a second electrode, and (b) measuring the alternating current impedance of the aspirated material between the first electrode and the second electrode. The method may further comprise identifying the aspirated material between the first electrode and the second electrode based upon the measured impedance. The method may be performed using one or more of the systems described herein.

### BRIEF DESCRIPTION OF THE DRAWINGS

The accompanying drawings illustrate implementations of the systems and methods disclosed herein and, together with the description, serve to explain the principles of the present disclosure.

FIG. 1 shows a schematic diagram of circuit components of an example system for identifying material aspirated during ophthalmic procedures in accordance with the disclosure.

FIG. 2 shows a cross-sectional view of an example of a vitrectomy instrument as part of a system for identifying aspirated material in accordance with the disclosure.

FIG. 3 shows a cross-sectional view of an example of an ultrasonic handpiece as part of a system for identifying aspirated material in accordance with the disclosure.

FIG. 4 shows a flowchart of steps in an example method of identifying material aspirated during an ophthalmic procedure in accordance with the disclosure.

The accompanying drawings may be better understood by reference to the following detailed description.

### DETAILED DESCRIPTION

For the purposes of promoting an understanding of the principles of the disclosure, reference will now be made to the implementations illustrated in the drawings, and specific language will be used to describe the same. It will nevertheless be understood that no limitation of the scope of the disclosure is intended. Any alterations and further modifications to the described systems, devices, instruments, methods, and any further application of the principles of the present disclosure are fully contemplated as would normally occur to one skilled in the art to which the disclosure relates. In particular, the features, components, and/or steps described with respect to one implementation may be combined with the features, components, and/or steps described with respect to other implementations of the disclosure. For simplicity, in some instances the same reference numbers are used throughout the drawings to refer to the same or like parts.

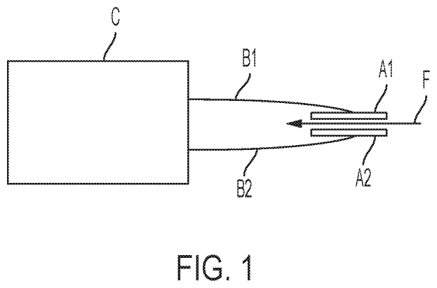

FIG. 1 shows a schematic diagram of circuit components of an example system for identifying materials aspirated during ophthalmic procedures in accordance with the disclosure. The circuit components are part of or form a circuit that provides high frequency (e.g., approximately 200 kHz to approximately 5 MHz), low voltage, non-physiologically destructive AC impedance measurement of materials aspirated from an eye during an ophthalmic procedure.

In the illustrated example, the circuit comprises two electrodes A1 and A2 arranged so that material aspirated from the eye flows between the two electrodes A1 and A2 in flow path F. The example circuit further includes an alternating current (AC) impedance measuring component C.

The term “component” as used herein is used broadly to embrace a group of electrical circuit elements connected together; the component may or may not itself comprise a circuit.

As shown in the schematic diagram of FIG. 1, the electrode A1 is connected to the AC impedance measuring component C by lead line B1, and the electrode A2 is connected to the AC impedance measuring component C by lead line B2.

In the illustrated circuit, the two electrodes A1 and A2 together form a structure through which aspirated material may flow. In the illustrated example, the first electrode A1 comprises a first flat plate and the second electrode A2 comprises a second flat plate parallel to the first flat plate. Alternatively, the electrodes may take other shapes or configurations, such as one electrode in the form of a tube and the other electrode in the form of a probe within the tube, wherein aspirated materials to be evaluated flow between the two electrodes.

In a system incorporating an electrode pair and AC impedance measuring component such as that shown in FIG. 1 or as otherwise described herein, the electrode pair may be incorporated into an ophthalmic instrument that is used for aspiration during an ophthalmic procedure. For example, the electrode pair may be placed in the flow path near the tip of a vitrectomy instrument, a phacofragmentation handpiece, a phacoemulsification handpiece, an extrusion handpiece, or another aspirating handpiece. The electrode pair may be located inside the instrument itself as a part of the instrument itself, or, in alternative embodiments, the electrode pair may be located outside of the device, such as in an aspiration tube that connects the ophthalmic instrument to a control console.

The circuit components for AC impedance measurement in a system as disclosed herein (such as the circuit components of FIG. 1) may be added to or may comprise circuit components of a standard diathermy or coagulation surgical module. Certain current ophthalmic systems, such as the CONSTELLATION® Vision System available from Alcon Laboratories, Inc. (Fort Worth, Tex.) or the CENTURION® Vision System available from Alcon Laboratories, Inc. (Fort Worth, Tex.), have modules for performing bipolar diathermy or coagulation. The circuit components for AC impedance measurement in a system as disclosed herein may be integrated with (or may comprise) circuit components of an existing module. For example, one electrode of the impedance measuring pair of electrodes (such as electrode A1 in FIG. 1) may be electrically connected to one of the electrodes of an existing bipolar diathermy/coagulation output, and the other electrode of the impedance measuring pair of electrodes (such as electrode A2 in FIG. 1) may be electrically connected to the other electrode of the existing bipolar diathermy/coagulation output.

In use, an AC impedance measuring component (such as the component C in FIG. 1) is switched into the output path and excites the electrode pair (e.g., electrodes A1 and A2 in FIG. 1) with a low voltage (e.g., 2Vpp) and high frequency (e.g., approximately 200 kHz to approximately 5 MHz). The AC impedance measuring component (such as the component C in FIG. 1) may be an off-the-shelf integrated circuit (e.g., AD5933 or AD5934 available from Analog Devices, Inc. of Norwood, Mass.) or a custom circuit or component for AC impedance measurement. The AC impedance measuring component measures the impedance of the material flowing between the electrodes of the pair of electrodes (e.g., in flow path F in FIG. 1).

In an ophthalmic procedure, different materials may be aspirated from the eye during the procedure. Such materials include, for example, vitreous, BSS, lens material (fragmented and/or emulsified), oil, water, and air. These materials will yield different impedances when measured using a circuit such as the circuit shown in FIG. 1. Based upon the measured impedance, the system therefore can identify what material is being aspirated at any time during the procedure.

Based on the measured impedance, the system may provide an output to inform the operator in real time of the material being aspirated. The output may be on a visual display, such as on a video screen, or it may be by some other signal, such as by audio signals or by different movement resistances on a foot pedal.

As referenced above, the system may include an ophthalmic instrument that is used for aspiration during an ophthalmic procedure. The electrode pair may be incorporated into the ophthalmic instrument.

One example of a suitable ophthalmic instrument for use in a system or method as described herein is a vitrectomy instrument. Examples of vitrectomy instruments are described and shown, for example, in U.S. Pat. Nos. 5,176,628, 8,038,692, 9,381,114, 9,615,969, and 9,757,273, the disclosures of which are hereby incorporated by reference herein in their entirety. An example of an available vitrectomy instrument is the ULTRAVIT® vitrectomy instrument, available from Alcon Laboratories, Inc. of Fort Worth, Tex.

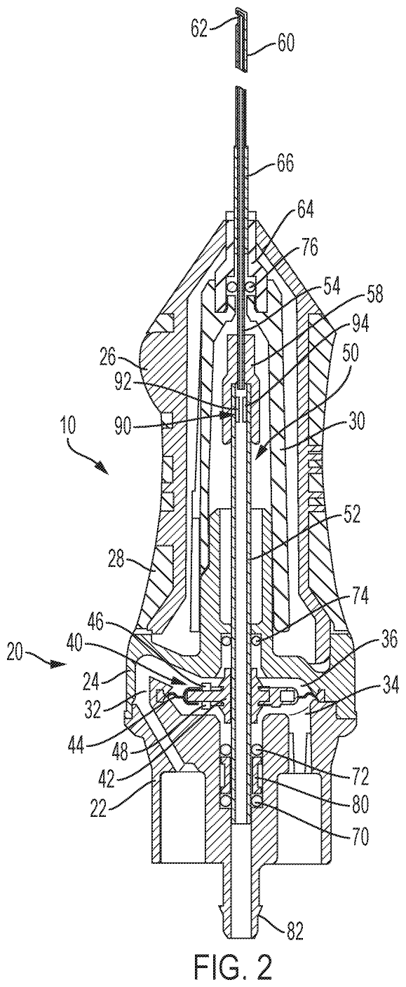

FIG. 2 is an example of a vitrectomy instrument 10 similar to the ULTRAVIT® vitrectomy instrument, with an electrode pair 90, for use in an example system as described herein. The vitrectomy instrument 10 comprises a housing 20 having a proximal end and a distal end. The housing 20 comprises a rear engine housing 22, a front engine housing 24, a first forward housing part 26, and a second forward housing part 28. The first and second forward housing parts 26, 28 may be made of different materials. For example, the first forward housing part 26 may be made of a rigid material, and the second forward housing part 28 may be made of a soft material to facilitate gripping. The vitrectomy instrument 10 also includes a retainer 30, that, in addition to the housing 20, serves to help position and guide the needle 60 and reciprocating cutter 50.

The vitrectomy instrument 10 has an engine comprising a diaphragm 40 that has a rigid diaphragm part 42 and a flexible membrane 44. The diaphragm 40 is located within an engine chamber 36. A first pneumatic passage 32 accesses a distal side of the diaphragm 40, and a second pneumatic passage 34 accesses a proximal side of the diaphragm 40. The diaphragm 40 includes a forward diaphragm/cutter stop 46 and a backward diaphragm/cutter stop 48.

The vitrectomy instrument 10 includes a needle 60 projecting from the distal end of the housing 20. The needle 60 has a proximal end and a distal end and a port 62 at its distal end. The needle is connected to a needle stiffener sleeve 66 which is bonded to a needle holder 64. The needle holder 64 is bonded to the retainer 30.

The vitrectomy instrument 10 also includes a reciprocating cutter 50. The reciprocating cutter 50 includes a cutter tube 54 that extends inside of the needle 60. The cutter tube 54 has a cutting edge at its distal end adapted to cut tissue drawn into the port 62. In the vitrectomy instrument 10 of FIG. 2, the reciprocating cutter 50 also includes a drive shaft 52 coupled to the cutter tube 54 by a coupling 58. The diaphragm 40 is connected to the reciprocating cutter 50 within the engine chamber 36.

The vitrectomy instrument 10 also includes several elastomeric o-rings 70, 72, 74, 76 that seal off various areas within the vitrectomy instrument 10. A spacer 80 is located between o-rings 70 and 72 and helps keep them in position. A vent (not shown) may be provided in the housing 20 adjacent the spacer 80 to allow venting of excess pressure.

In use of the vitrectomy instrument 10, the needle 60 is inserted into the eye of a patient with the port 62 adjacent tissue to be removed, e.g., vitreous fibers. With suction applied through the cutter tube 54, the cutter tube 54 is caused to reciprocate at high speed with the cutting edge moving back and forth across the port 62. The reciprocating motion is caused by pneumatic actuation via the pneumatic passages 32, 34 causing the diaphragm 40 to move back and forth at high speed. Because the diaphragm 40 is connected to the reciprocating cutter 50, the back and forth movement of the diaphragm 40 causes reciprocating motion of the reciprocating cutter 50. The suction through the cutter tube 54 acts through the port 62 to draw vitreous fibers into the port 62. As the cutter tube 54 moves distally across the port 62, the cutting edge severs the vitreous fibers so that they can be suctioned away and removed.

When the vitrectomy instrument 10 is in use, suction is applied from a console to which the vitrectomy instrument 10 is coupled by an aspiration tube (not shown). The aspiration tube connects to the proximal end of a coupling 82 and applies suction through the lumens of the coupling 82, the drive shaft 52, the cutter tube 54, and the end of the needle 60 through the port 62. These components together form an aspiration path through which aspirated material flows away from the eye during the ophthalmic procedure.

As shown in FIG. 2, the vitrectomy instrument 10 also includes an electrode pair 90 comprising a first electrode 92 and a second electrode 94, wherein the electrode pair 90 is located in the aspiration path. In this illustrated example, the first electrode 92 and the second electrode 94 are positioned inside the drive shaft 52 of the reciprocating cutter 50, although other locations are possible. For example, the first electrode 92 and the second electrode 94 may be positioned inside the lumen of the coupling 82 or inside the aspiration tube.

The first electrode 92 and the second electrode 94 are similar to the electrodes A1 and A2 in FIG. 1. As described with respect to FIG. 1, the two electrodes 92 and 94 are arranged so that material aspirated from the eye flows between the two electrodes 92 and 94. The electrode 92 is connected to an AC impedance measuring component by a lead line (not shown), and the electrode 94 is connected to the AC impedance measuring component by lead line (not shown).

In use, the AC impedance measuring component is switched into the output path and excites the electrode pair 90 with a low voltage and high frequency. The AC impedance measuring component measures the impedance of the material flowing between the electrodes of the electrode pair 90. Based upon the measured impedance, the system identifies what material is being aspirated at any time during the procedure (e.g., vitreous, BSS, lens material (fragmented and/or emulsified), oil, water, and/or air). The system may provide an output (e.g., visual, audio, and/or tactile output) to inform the operator in real time of the material being aspirated.

Another example of a suitable ophthalmic instrument for use in a system or method as described herein is an ultrasonic handpiece, such as a phacoemulsification handpiece. Examples of ultrasonic handpieces (and working tips for ultrasonic handpieces) are described and shown, for example, in U.S. Pat. Nos. 3,589,363, 4,223,676, 4,246,902, 4,493,694, 4,515,583, 4,589,415, 4,609,368, 4,869,715, 4,922,902, 5,178,605, 6,402,769, 6,602,193, 7,572,242, 7,651,490, and 8,814,894, the disclosures of which are hereby incorporated by reference herein in their entirety. An example of an available ultrasonic handpiece is the OZIL® ultrasonic handpiece, available from Alcon Laboratories, Inc. of Fort Worth, Tex.

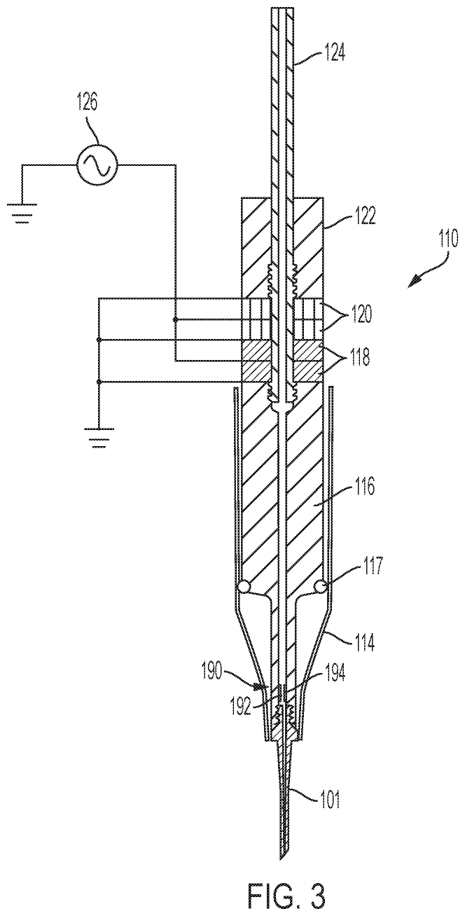

FIG. 3 is an example of an ultrasonic handpiece 110, with an electrode pair 190, for use in an example system as described herein. The handpiece 110 may be similar, for example, to that depicted in U.S. Pat. Nos. 6,402,769 or 7,651,490. The handpiece 110 may be similar, for example, to the OZIL® ultrasonic handpiece available from Alcon Laboratories, Inc. of Fort Worth, Tex. The handpiece 110 may be an ultrasonic handpiece that can produce torsional ultrasonic vibrations, longitudinal ultrasonic vibrations, or both. The mode of operation (torsional or longitudinal) and the frequency of the vibrations may be selected by operator input to a connected surgical console (not shown).

As can be seen in FIG. 3, the handpiece 110 has a handpiece shell 114, ultrasound horn 116, torsional ultrasound crystals 118, and longitudinal ultrasound crystals 120. Horn 116 is held within shell 114 by isolator 117. Crystals 118 and 120 are held within shell 114 and in contact with horn 116 by back cylinder 122 and bolt 124. Crystals 118 and 120 vibrate ultrasonically in response to a signal generated by ultrasound generator 126. Crystals 118 are polarized to produce torsional motion. Crystals 120 are polarized to produce longitudinal motion. In an alternative, as illustrated in FIG. 6 of U.S. Pat. No. 6,402,769, the handpiece may have one or more crystals used to produce both longitudinal and torsional motion. In another alternative, the handpiece may have crystals used to produce only longitudinal or only torsional motion.

The signal generated by ultrasound generator 126 may be controlled by an operator using the control system of surgical console 100. The signals from the surgical console are transmitted through the electrical cable 106 to the crystals 118 and 120 of the handpiece 110. Activation of the crystals causes ultrasonic movement of the horn 116, which in turn causes ultrasonic movement of an ultrasonic needle tip 101, such as a phacoemulsification needle tip that can be used to break up and aspirate a cataractous lens in cataract surgery.

When the ultrasonic handpiece 110 is in use, suction is applied from a console to which the ultrasonic handpiece 110 is coupled by an aspiration tube (not shown). The aspiration tube connects to the proximal end of the bolt 124 (or a coupling connected to the bolt 124) and applies suction through the lumens of the bolt 124, the horn 116, and the needle tip 110. These components together form an aspiration path through which aspirated material (e.g., fragmented and/or emulsified lens material) flows away from the eye during the ophthalmic procedure.

As shown in FIG. 3, the ultrasonic handpiece 110 also includes an electrode pair 190 comprising a first electrode 192 and a second electrode 194, wherein the electrode pair 190 is located in the aspiration path. In this illustrated example, the first electrode 192 and the second electrode 194 are positioned inside the ultrasonic horn 116, although other locations are possible. For example, the first electrode 192 and the second electrode 194 may be positioned inside the lumen of the bolt 124 or inside the aspiration tube.

As with the electrodes 92 and 94, the first electrode 192 and the second electrode 194 are similar to the electrodes A1 and A2 in FIG. 1. The two electrodes 192 and 194 are arranged so that material aspirated from the eye flows between the two electrodes 192 and 194. The electrode 192 is connected to an AC impedance measuring component by a lead line (not shown), and the electrode 194 is connected to the AC impedance measuring component by lead line (not shown).

In use, the AC impedance measuring component is switched into the output path and excites the electrode pair 190 with a low voltage and high frequency. The AC impedance measuring component measures the impedance of the material flowing between the electrodes of the electrode pair 190. Based upon the measured impedance, the system identifies what material is being aspirated at any time during the procedure (e.g., vitreous, BSS, lens material (fragmented and/or emulsified), oil, water, and/or air). The system may provide an output (e.g., visual, audio, and/or tactile output) to inform the operator in real time of the material being aspirated.

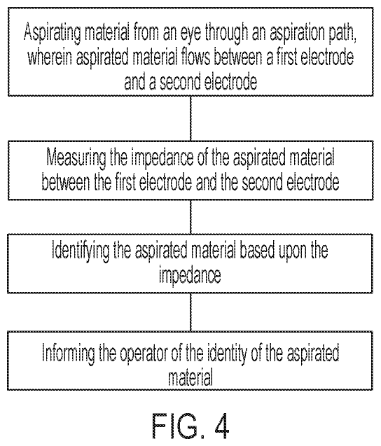

FIG. 4 shows a flowchart of steps in an example method of identifying material aspirated during an ophthalmic procedure in accordance with the disclosure. A first step comprises aspirating material from an eye through an aspiration path, wherein aspirated material flows between a first electrode and a second electrode. The flow path may comprise any of the components of an instrument through which aspirated material flows, an aspiration tube, and/or console components or other components through which aspirated material flows. The first and second electrodes may be located anywhere along the flow path, including in the instrument itself and/or in the aspirating tube.

A second step comprises measuring the impedance of the aspirated material between the first electrode and the second electrode. This step may be performed using an AC impedance measuring component or device as described above.

A third step comprises identifying the aspirated material based upon the impedance. Because different materials will have different measured impedances, the measured impedance can be used to identify the aspirated material.

A fourth step comprises informing the operator of the identity of the aspirated material. This may be done by visual output (e.g., on a display), audio output, and/or tactile output (e.g., by changing the resistance on a button or foot pedal).

The first and second electrodes as disclosed herein may have other uses as well. For example, the structure may allow fluid path pressure sensing very close to the eye. This may allow the ability to sense intraocular pressure (IOP) much closer to the eye. If this information is used to control infusion, it may allow the fluid control system to close the control loop right at the eye, eliminating the negative effects of the fluidics cassette and tubing sets, further improving chamber stability and overall fluidics performance. For example, the pair of electrodes may be arranged in a device similar as described above, with one or both of the electrodes being movable (deflectable) based on the pressure of the infusion material between them. In this example, the pair of electrodes together with the infusion material between them form a capacitor. If desired, the electrodes may be larger than those used in the above examples. The impedance characteristics of the infusion material (e.g., BSS or BSS+) are known. The difference in pressures will cause small movement (deflection) of the moveable electrode(s), pushing the electrodes apart under higher pressure, and drawing them nearer under lower pressures. Because the capacitance in a parallel plate capacitor is proportional to the distance between the plates, measuring the AC impedance as described above enables the calculation of the distance between the plates, from which the pressure can then be determined.

With respect to the systems and methods as disclosed herein for identifying aspirated material, these systems and methods may have one or more advantages. For example, the electrodes and capacitors as described herein are inexpensive, rendering the resulting systems inexpensive. As discussed above, the systems and methods may make use of an existing or standard diathermy/coagulation surgical module, reducing cost and making the systems and methods relatively simple to implement. The systems and methods may be cost-effective, requiring only the integration of small electrodes (e.g., parallel plate electrodes) in the fluid path and an inexpensive connection to the diathermy/coagulation module. The addition to the diathermy/coagulation module that measures the AC impedance is inexpensive as well. The systems and methods are safe, as they may be based on the same or similar energy frequencies as are already used by diathermy/coagulation inside the eye.

Being able to distinguish between materials in real time will allow the operator to facilitate a more accurate, more complete, more efficient, and/or shorter vitrectomy, cataract procedure, or other procedure, potentially leading to better long-term outcomes. Currently, it can be difficult for a person performing an ophthalmic procedure to know what type of material is being aspirated from the eye at any given time during the procedure. For example, vitreous material and BSS are optically similar by design, so it is difficult to distinguish the two by visualization. Alternating current (AC) impedance measurement provides advantages over direct current (DC) impedance measurement, because in certain instances DC impedance measurement has difficulty in distinguishing between different materials (such as between vitreous and BSS), whereas with AC, at certain frequencies there are significant enough differences in impedances of the different materials being aspirated that the system is capable of distinguishing between them. The systems and methods described herein can improve procedures and potentially patient outcome.

Persons of ordinary skill in the art will appreciate that the implementations encompassed by the disclosure are not limited to the particular exemplary implementations described above. In that regard, although illustrative implementations have been shown and described, a wide range of modification, change, and substitution is contemplated in the foregoing disclosure. It is understood that such variations may be made to the foregoing without departing from the scope of the disclosure. Accordingly, it is appropriate that the appended claims be construed broadly and in a manner consistent with the disclosure.

What is claimed is:

1. A system for identifying material aspirated during an ophthalmic procedure, the system comprising:
an aspiration path through which aspirated material flows away from the eye during the ophthalmic procedure; and
a circuit comprising:
a first electrode and a second electrode, wherein the first electrode and the second electrode are positioned such that aspirated material flows between the first electrode and the second electrode when the aspirated material flows away from the eye; and
an alternating current impedance measuring component connected to the first electrode and the second electrode, wherein the alternating current impedance measuring component is configured to measure the impedance of the aspirated material between the first electrode and the second electrode in order to identify the aspirated material.

2. A system for identifying material aspirated during an ophthalmic procedure as in claim 1, wherein the first electrode comprises a first flat plate and the second electrode comprises a second flat plate parallel to the first flat plate.

3. A system for identifying material aspirated during an ophthalmic procedure as in claim 1, wherein the first electrode comprises a tube and the second electrode comprises a probe inside of the tube.

4. A system for identifying material aspirated during an ophthalmic procedure as in claim 1, wherein the circuit is configured to supply alternating current through the electrodes.

5. A system for identifying material aspirated during an ophthalmic procedure as in claim 1, wherein the circuit is configured to supply alternating current through the electrodes at a frequency from approximately 200 kHz to approximately 5 MHz.

6. A system for identifying material aspirated during an ophthalmic procedure, the system comprising:
an ophthalmic instrument for performing an ophthalmic procedure, the ophthalmic instrument comprising an aspiration path through which aspirated material flows away from the eye during the ophthalmic procedure; and
a circuit comprising:
a first electrode and a second electrode, wherein the first electrode and the second electrode are positioned such that aspirated material flows between the first electrode and the second electrode when the aspirated material flows away from the eye; and
an alternating current impedance measuring component connected to the first electrode and the second electrode, wherein the alternating current impedance measuring component is configured to measure the impedance of the aspirated material between the first electrode and the second electrode in order to identify the aspirated material.

7. A system for identifying material aspirated during an ophthalmic procedure as in claim 6, wherein the ophthalmic instrument comprises a vitrectomy instrument.

8. A system for identifying material aspirated during an ophthalmic procedure as in claim 6, wherein the ophthalmic instrument comprises an ultrasonic handpiece.

9. A system for identifying material aspirated during an ophthalmic procedure as in claim 6, wherein the ophthalmic instrument comprises a phacofragmentation handpiece.

10. A system for identifying material aspirated during an ophthalmic procedure as in claim 6, wherein the ophthalmic instrument comprises a phacoemulsification handpiece.

11. A system for identifying material aspirated during an ophthalmic procedure as in claim 6, wherein the ophthalmic instrument comprises an aspirating handpiece.

12. A system for identifying material aspirated during an ophthalmic procedure as in claim 6, wherein the ophthalmic instrument comprises an extrusion handpiece.

13. A system for identifying material aspirated during an ophthalmic procedure as in claim 6, wherein the first electrode and the second electrode are part of the ophthalmic instrument.

14. A system for identifying material aspirated during an ophthalmic procedure as in claim 6, wherein the first electrode and the second electrode are positioned outside of the ophthalmic instrument.

15. A system for identifying material aspirated during an ophthalmic procedure as in claim 6, wherein the first electrode and the second electrode are positioned inside of an aspiration tube.

16. A method of identifying material aspirated during an ophthalmic procedure, the method comprising:
aspirating material from an eye through an aspiration path, wherein aspirated material flows between a first electrode and a second electrode; and
measuring the alternating current impedance of the aspirated material between the first electrode and the second electrode.

17. A method of identifying material aspirated during an ophthalmic procedure as in claim 16, further comprising the step of identifying the aspirated material between the first electrode and the second electrode based upon the measured impedance.

18. A method of identifying material aspirated during an ophthalmic procedure as in claim 16, wherein the step of measuring the impedance of the aspirated material between the first electrode and the second electrode comprises supplying alternating current through the electrodes.

19. A method of identifying material aspirated during an ophthalmic procedure as in claim 16, wherein the first electrode and the second electrode are part of an ophthalmic instrument.

20. A method of identifying material aspirated during an ophthalmic procedure as in claim 16, wherein the first electrode and the second electrode are positioned outside of an ophthalmic instrument.
